(19) United States
(12) Patent Application Publication
(10) Pub. No.: US 2024/0290496 A1
(43) Pub. Date: Aug. 29, 2024

(54) ARTIFICIAL INTELLIGENCE BASED SYSTEMS AND METHODS FOR SITUATIONAL MENTAL HEALTH CONDITION PREDICTIONS AND SUPPORT

(71) Applicant: Accenture Global Solutions Limited, Dublin (IE)

(72) Inventors: Ramandeep Kaur, Gurgaon (IN); Gagandeep Singh, New Delhi (IN)

(21) Appl. No.: 18/175,653

(22) Filed: Feb. 28, 2023

(51) Int. Cl. G16H 50/30; G16H 50/20

(52) U.S. Cl. CPC G16H 50/30; G16H 50/20

(57) ABSTRACT

Systems and methods for predicting a person's likelihood of a situational mental health condition (such as post-partum depression (PPD)) diagnosis using machine learning models are described. In one example, a mental health risk assessment system receives data at recurring intervals for a person during their pregnancy and in the year after their pregnancy. Based on both the recurring data for the person as well as the specific time period at which the data is obtained, a machine learning model can generate highly accurate mental health predictions. The mental health risk assessment system can further implement a software application that manages the flow of information from patients as well as presentation of information to the patients. Furthermore, the application can present targeted information for a patient to the patient's doctor or other medical personnel/facility.

### TECHNICAL FIELD

The present disclosure generally relates to the field of predictive modeling of a situational mental health condition (e.g., postpartum depression). More specifically, the present disclosure generally relates to the use of predictive models to monitor a person's likelihood of exhibiting symptoms of a situational mental health condition (e.g., postpartum depression) using time-bound machine learning models.

### BACKGROUND

Life events, such as pregnancy and birth have a significant impact on mental health. For example, postpartum depression (PPD), defined as having an episode of minor or major depression during pregnancy or up to one year after giving birth, is a relatively common condition that affects 1 in 4 new mothers. The etiology of PPD is not well understood, but the condition likely arises from a combination of psychological, psychosocial, and biological factors, making it difficult to diagnose. Well-documented but complex biological risk factors for post-partum mental health issues, such as PPD, are hypothalamic-pituitary-adrenal axis dysregulation, inflammatory processes, genetic vulnerability, and allopregnanolone withdrawal. Psychosocial factors include previous depression, severe life events, chronic stress, and relationship struggles. The role of resilience and personality have been lately also gaining attention.

Mental health disorders during pregnancy and the year following birth can negatively impact a mother's life and can also affect a child's development by interfering with the mother-infant relationship. For instance, children of mothers with PPD have greater cognitive, behavioral, and interpersonal problems compared to children of mothers without PPD. Despite PPD being a detrimental health condition for many women, numerous affected women fail to receive adequate care. Effective treatments and interventions exist for mental health disorders, but they are cost-effective only among high-risk women. Traditional methods attempt to predict women at risk by prenatal assessment of critical variables. However, to date, there has been no effective way to predict women at risk for the development of depressive symptoms postpartum.

Obstetric and pediatric practitioners must have confidence that screening processes accurately identify depression among the women in their diverse practices. Since depression or other mental health conditions can occur anytime in the postpartum year, screening only in the early postpartum period is insufficient.

There is a need in the art for a system and method that addresses the shortcomings discussed above.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like reference numerals designate corresponding parts throughout the different views.

FIG. 1 is a schematic diagram of a high-level overview of a post-partum depression (PPD) detection framework, according to an embodiment;

FIG. 2 is a schematic diagram of an environment for a mental health risk assessment system, according to an embodiment;

FIG. 3 is a schematic diagram listing categories for data that can be provided on behalf of the patient for ingestion by the mental health risk assessment system, according to an embodiment;

FIG. 4 is a schematic diagram showing the development and implementation of a machine learning prediction model for PPD, according to an embodiment;

FIG. 5 is a table showing a timespan across which multiple data entries are provided to correspond to pre-designated time periods within the timespan, according to an embodiment;

FIG. 6 is an example of a user interface for an application of the mental health risk assessment system configured for use by a doctor, according to an embodiment;

FIG. 7 is an example of a user interface for an application of the mental health risk assessment system configured for use by a patient, according to an embodiment;

FIG. 8 is an example of a user interface for an application of the mental health risk assessment system configured for use by a medical facility, according to an embodiment;

FIG. 9 is a flow chart depicting a method for predicting a post-partum depression (PPD) diagnosis, according to an embodiment; and

FIG. 10 is a diagram depicting example environments and components by which systems and/or methods, described herein, may be implemented.

### SUMMARY

Implementations described herein provide for a mental health risk assessment system that can generate predictions regarding the likelihood of a person experiencing a mental health disorder during or after experiencing a life event. For example, the mental health disorder may be postpartum depression (PPD) and the prediction may be made during pregnancy or in the year following the pregnancy. The system can automatically generate notifications and/or alerts to designated caretakers such as obstetricians, primary care doctors, psychiatrists, therapists, and other healthcare providers or family members of the patient that can help ensure mental health treatment is provided to the patient in a timely and personalized approach. For example, the system can receive data at recurring intervals for a person during their pregnancy and in the year after their pregnancy. Based on both the recurring data for the person as well as the specific time period at which the data is obtained, a machine learning model can generate highly accurate mental health predictions. The model can be re-trained and improved over time as additional training and patient data, including the result of receiving recommended treatment, is obtained. In some embodiments, the system applies time-bound rules that vary the weights assigned to the array of variables during different stages of time across the pregnancy and the year following.

In one aspect, the disclosure provides a computer-implemented method for predicting a mental health condition (e.g., postpartum depression) during and/or after a life event (e.g., pregnancy). The method includes steps of (1) receiving, at a mental health risk assessment system, a first training dataset including data representing women during and in the year following pregnancy; (2) training, using the first training dataset, a mental health condition prediction machine learning (ML) model to generate mental health risk predictions based on one or more time-bound rules; (3) receiving, at a mental health risk assessment system, a first set of data representing information about a first patient during a first time, the first set of data including a first value for a first variable and a second value for a second variable; (4) determining the first time corresponds to a first pre-designated period that falls in an assessment timespan when a person is either pregnant or within a year after experiencing childbirth; (5) selecting a first time-bound rule based on the first pre-designated period; (6) assigning, at the mental health condition prediction ML model and based on the first time-bound rule, a first weight to the first value and a second weight to the second value; (7) generating, via the mental health condition prediction ML model, a first prediction for the first patient based on the first time-bound rule as applied to the first set of data; and (8) automatically transmitting a notification describing the first prediction to one or more health personnel associated with the first patient.

In another aspect, the disclosure provides a non-transitory computer-readable medium storing software comprising instructions executable by one or more computers which, upon such execution, cause the one or more computers to predict a mental health condition during and/or after pregnancy by performing the following: (1) receive, at a mental health risk assessment system, a first training dataset including data representing women during and in the year following pregnancy; (2) train, using the first training dataset, a mental health condition prediction machine learning (ML) model to generate mental health risk predictions based on one or more time-bound rules; (3) receive, at a mental health risk assessment system, a first set of data representing information about a first patient during a first time, the first set of data including a first value for a first variable and a second value for a second variable; (4) determine the first time corresponds to a first pre-designated period that falls in an assessment timespan when a person is either pregnant or within a year after experiencing childbirth; (5) select a first time-bound rule based on the first pre-designated period; (6) assign, at the mental health condition prediction ML model and based on the first time-bound rule, a first weight to the first value and a second weight to the second value; (7) generate, via the mental health condition prediction ML model, a first prediction for the first patient based on the first time-bound rule as applied to the first set of data; and (8) automatically transmit a notification describing the first prediction to one or more health personnel associated with the first patient.

In another aspect, the disclosure provides a system for predicting a mental health condition (e.g., postpartum depression) during and/or after a life event (e.g., pregnancy). The system comprises one or more computers and one or more storage devices storing instructions that are operable, when executed by the one or more computers, to cause the one or more computers to: (1) receive, at a mental health risk assessment system, a first training dataset including data representing women during and in the year following pregnancy; (2) train, using the first training dataset, a mental health condition prediction machine learning (ML) model to generate mental health risk predictions based on one or more time-bound rules; (3) receive, at a mental health risk assessment system, a first set of data representing information about a first patient during a first time, the first set of data including a first value for a first variable and a second value for a second variable; (4) determine the first time corresponds to a first pre-designated period that falls in an assessment timespan when a person is either pregnant or within a year after experiencing childbirth; (5) select a first time-bound rule based on the first pre-designated period; (6) assign, at the mental health condition prediction ML model and based on the first time-bound rule, a first weight to the first value and a second weight to the second value; (7) generate, via the mental health condition prediction ML model, a first prediction for the first patient based on the first time-bound rule as applied to the first set of data; and (8) automatically transmit a notification describing the first prediction to one or more health personnel associated with the first patient.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

While various embodiments are described, the description is intended to be exemplary, rather than limiting, and it will be apparent to those of ordinary skill in the art that many more embodiments and implementations are possible that are within the scope of the embodiments. Although many possible combinations of features are shown in the accompanying figures and discussed in this detailed description, many other combinations of the disclosed features are possible. Any feature or element of any embodiment may be used in combination with or substituted for any other feature or element in any other embodiment unless specifically restricted.

This disclosure includes and contemplates combinations with features and elements known to the average artisan in the art. The embodiments, features, and elements that have been disclosed may also be combined with any conventional features or elements to form a distinct invention as defined by the claims. Any feature or element of any embodiment may also be combined with features or elements from other inventions to form another distinct invention as defined by the claims. Therefore, it will be understood that any of the features shown and/or discussed in the present disclosure may be implemented singularly or in any suitable combination. Accordingly, the embodiments are not to be restricted except in light of the attached claims and their equivalents. Also, various modifications and changes may be made within the scope of the attached claims.

### DESCRIPTION OF EMBODIMENTS

The proposed systems and methods are directed to data analysis using machine learning models with the goal of accurately predicting a person's mental state (e.g., likelihood of experiencing postpartum depression) in order to proactively manage and treat their symptoms. While the discussed embodiments focus on postpartum depression, it is understood that the disclosed systems and methods may apply to other areas of mental health. For example, the disclosed systems and methods may apply to a patient's mental health after a large life event, such as marriage, divorce, moving to a new area, loss of job, family death, etc., and may apply to a period of time relative to the life event.

As a general matter, the time following the birth of a child is one of intense physiological and psychological change for new mothers. The process of pregnancy and childbirth represents such a stressful life event that many women experience the onset of depressive episodes. This period has been associated with significant negative effects not only on depressed women themselves, but on the physical, cognitive, and emotional development of their children. Medical professionals—including obstetricians and pediatricians—can serve important roles in screening for and treating postpartum depression. There is a need for a greater appreciation of the importance of early and accurate detection and treatment of PPD during and after pregnancy. Early detection and intervention greatly decrease its risks.

A continuous, real-time screening tool and monitoring service for postpartum depression is described below. This tool, being conveyed and shared across a digital medium (e.g., mobile applications) can represent a cost-effective, convenient, and standardized screening instrument. In different embodiments, this tool implements a specialized machine learning (ML) model that is trained to accurately predict the likelihood of a woman experiencing PPD during her pregnancy and the year following. The model can leverage the power of clinical, biochemical, demographic, and psychometric data, collected both during and post-pregnancy and be used to empower patients and medical personnel with individualized diagnosis, prognosis, and treatment options for PPD. As will be illustrated herein, the predictions and associated information can be presented via a software application (“app”) that is configured to offer multiple and dynamic user interface formats that are targeted to the designated user/audience type (e.g., doctor, nurse, patient, lab, hospital, etc.). Furthermore, the app can be hardware and platform-agnostic, can be configured with multi-language support, and can incorporate multi-head attention mechanisms for parallel processing and enhanced computation. The proposed systems can thereby facilitate precise, early detection of PPD, as well as timely and apt interventional recommendations, not only at the individual patient level, but scaled up to service larger segments in underserved rural areas or even regional or national level initiatives.

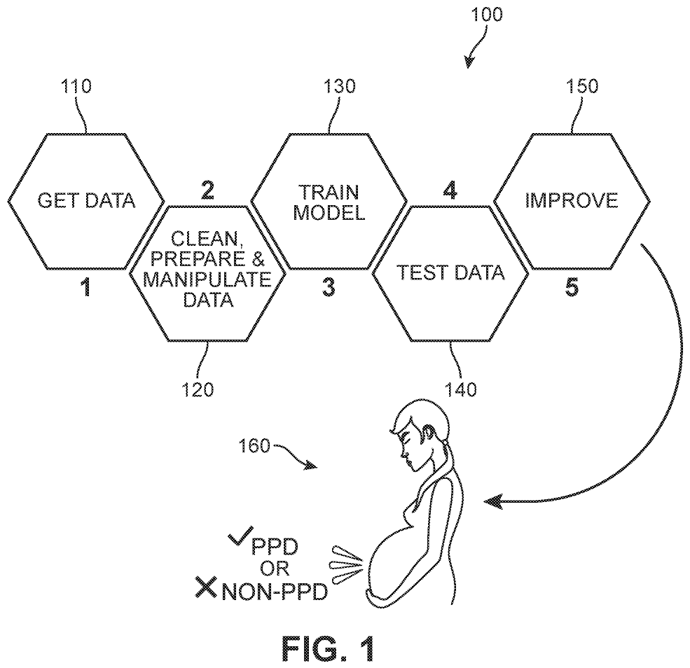

Referring to FIG. 1, an overview of the proposed embodiments is depicted. As shown in FIG. 1, a mental health detection framework 100 can collect data related to patients' pregnancy and post-pregnancy condition in a first stage 110. The data can be cleaned, pre-processed, and manipulated in a second stage 120. In some embodiments, the data can comprise training data that is used to train a machine learning prediction model of a mental health risk assessment (detection) system in a third stage 130. The data and results can be tested in a fourth stage 140, and monitored and/or updated to enable a continuous improvement of the prediction in a fifth stage 150. The trained model can then receive individual user data, analyze the data, and generate predictions and based on the data, including a prediction 160 regarding the user's predilection for PPD or other mental health illness. In some embodiments, in the cases where there is a high likelihood of such a diagnosis, the system can further select one or more specific interventional recommendations (also referred to herein as “interventions”) that have been shown to be effective for someone of the user's demographics and other specific characteristics that can be presented to the doctor, for example.

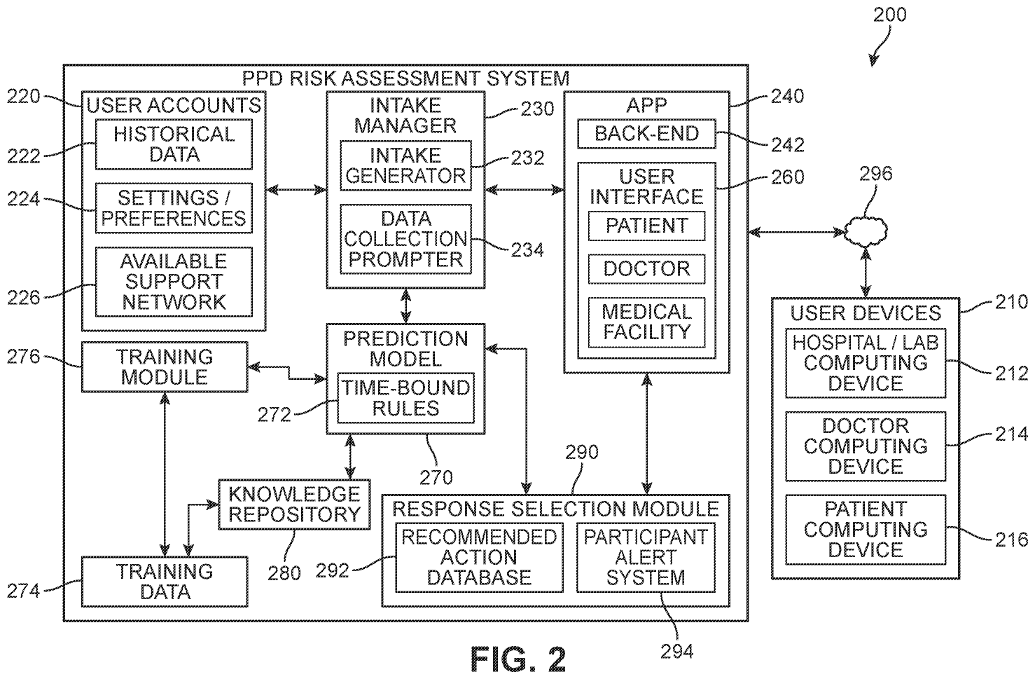

In order to provide the reader with a greater appreciation of the embodiments, FIG. 2 depicts an overview of an embodiment of an environment 200 for implementation of a mental health risk assessment system 250 (or system 250), also referred to as a PPD detection system and/or PPD prediction system, although throughout this disclosure it should be understood the use of the terminology “PPD” will encompass any mental disorder, illness, or condition that can occur during a woman's pregnancy or in the year after pregnancy, rather than being limited to the PPD diagnosis. As shown in FIG. 2, the system 250 operates in conjunction with inputs and selections made by various types (or classes) of end-users via their user devices 210, such as hospitals or labs (represented by a hospital/lab computing device 212), doctors and other medical professionals/personnel (represented by a doctor computing device 214), and patients (represented by a patient computing device 216). In other embodiments, end-users can also include family and friends of the patient, and/or mental health professionals. These end-users can interact with modules of system 250 over a network 296.

In different embodiments, network 296 could include one or more Wide Area Networks (WANs), Wi-Fi networks, Bluetooth or other Personal Area Networks, cellular networks, as well as other kinds of networks. It may be appreciated that different devices could communicate using different networks and/or communication protocols. The devices can include computing or smart devices as well as simpler IoT devices configured with a communications module/interface and a sensor. The communication module may include a wireless connection using Bluetooth® radio technology, communication protocols described in IEEE 802.11 (including any IEEE 802.11 revisions), Cellular technology (such as GSM, CDMA, UMTS, EV-DO, WiMAX, or LTE), or Zigbee® technology, among other possibilities. In many cases, the communication module is a wireless connection; however, wired connections may also be used. For example, the communication module may include a wired serial bus such as a universal serial bus or a parallel bus, among other connections. In addition, each device can include provisions for communicating with, and processing information from, system 250. Each device may include one or more processors and memory. Memory may comprise a non-transitory computer readable medium. Instructions stored within memory may be executed by the one or more processors.

As shown in FIG. 2, in different embodiments, the system 250 can include one or more modules and/or components, including a user accounts database 220, an intake manager 230, application (“app”) 240, a knowledge repository 280, a prediction model 270, a training module 276, and a response selection module 290. In some embodiments, the knowledge repository 280 stores “scrubbed” (i.e., personal identifier information removed) data for a set of individuals that can be used as training and testing data 274 for training of the prediction model 270 by training module 276. In one example, the training module 276 splits the data in the knowledge repository into two segments (training and testing), for example, where 80% of the data is used as training data and 20% is used as testing data. It can be appreciated that as additional subject data is fed into the knowledge repository 280, and/or as the data that is received is more nuanced and representative of the weighted parameters on which the prediction model 270 relies, the decisions made by the prediction model 270 can become increasingly accurate and robust.

In one example, the training module 276 incorporates a digital matrix of time-bound variables that is created and stored in the database (e.g., knowledge repository 280). The training module 276 processes the anonymized data and develops the prediction model 270 as a time-bound model that dynamically evaluates a patient's likely PPD (or other mental health condition) occurrence based on historical patient data during their pregnancy journey. The training and testing data 274 can be built using the data in the knowledge repository 280 and stored within the system securely.

In some embodiments, the system 250 can generate data sets from the data stored in the knowledge repository 280, including training and testing data 274. Generally, a data set may be a set of multiple data objects, and a training set may be a data set of data objects used for inferring a function for classification (i.e., a classifier). The training sets may include supervised training sets that include labeled data objects, which are used by one or more machine learning functions to generate the classifiers. Each of the labels for the data objects may indicate whether the respective data object is classified under a particular category. Labels may be manually generated, may be specified in historic data, or may be generated automatically.

For example, the data in an example, during a training phase, the training sets are input into the machine learning functions. A machine learning function being used to train a classifier may adjust parameters in the classifier so that it makes accurate predictions for the training set. The machine learning functions 04 may include a known induction algorithm, such as Naive Bayes, C4.5 decision trees, Support Vector Machines, logistic regression, step-wise logistic regression, chi-squared tests for predictive variable selection, and others. Accordingly, inputting a training set to a machine learning function may generate a classifier, such as one of the classifiers, trained to classify the data objects into a category associated with the labels in the training set. After being trained, the classifiers may be used to classify data objects without labels, such as data objects.

Furthermore, it should be appreciated that as more patients are enrolled with the service, their own data as well as any PPD or other mental health diagnosis, can be scrubbed for personal identifier information and added to the knowledge repository to provide a continuous training/testing and source for optimization and improvement of the prediction model 270. The patient can also update their settings/preferences 224 to indicate how they want the system 250 to handle their data (e.g., it should or should not be added to the knowledge repository, should be deleted, etc.), and the types and frequency of notifications they want to receive from the app 240.

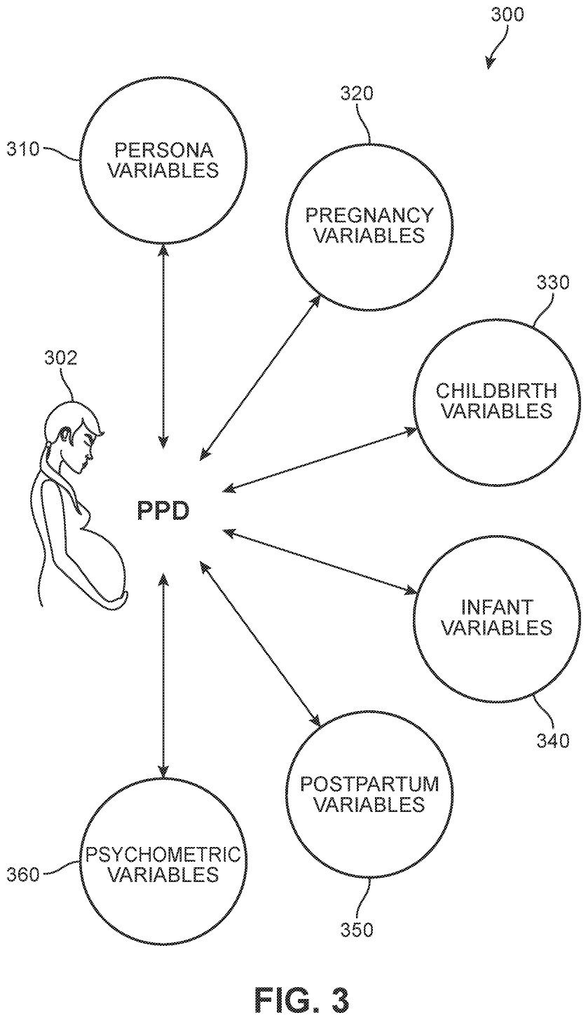

In different embodiments, the information stored in the knowledge repository 280 can reflect a wide range of data that may be optionally classified as falling into one or more categories pertinent to the implementation and operation of the prediction model 270, which are described with reference to FIG. 3. In addition, as shown in FIG. 2, as data is collected from individual end-users (patients), stored as historical patient data 222 (also referred to herein as historical data) under the user's profile in the user accounts database 220, the incoming data can be similarly classified.

Thus, in different embodiments, the prediction model relies on variable classes that have been pre-defined (e.g., using programming languages such as Python, NodeJS, Ruby, Java, PHP, Golang, etc.). Furthermore, in different embodiments, each variable is assigned a weight that can vary from week to week (or other dynamic time period). In other words, the importance or influence of a specific factor (e.g., the woman's HbA1c level) can play a greater role in the detection of PPD or another mental illness, such as anxiety, etc. at week 20 of pregnancy so can be assigned a ‘heavier’ or larger weight in the prediction model 270 during week 20 during the pregnancy, but the same factor may be less important in determining the woman's likelihood of PPD at week 9 after pregnancy. Thus, one or more variables can be time-bound, and assigned weights that vary from one time period to another based on time-bound rules 272, thereby optimizing the accuracy of the PPD prediction. These time-bound rules 272 may be adjusted from time to time to modify the distribution of weights at a particular window of time as more training and testing data 274 is received and the effects of each variable can be more precisely defined.

In some embodiments, the prediction engine for the model is configured to predict a closest probability match for the present, as well as a future PPD (or other mental health concern) probability prediction for the given unique (but anonymized) patient case ID. In some embodiments, the prediction model 270 resides on and runs on a central server that is independent of the front-end aspects for app 240. Furthermore, in different embodiments, the system 250 is optimized to allow for multi-threading at the central server to enable catering to multiple mobile apps and devices for patients simultaneously. Once a prediction is generated by the prediction model 270, the output can be shared with a response selection module 290, which will determine how and to whom the app 240 will present the decision, as discussed in further detail below.

As a general matter, the prediction model 270 can incorporate machine learning (ML) techniques, or model learning through data relationships. This approach removes user bias and preconceived notions of system dynamics and behavior and enables the data to describe the complex relationships among inputs, or from inputs to outputs. The ability to model a diagnosis for underlying patient behavior and physical measurements and their relationships enables the system to estimate the patient's expected condition and to forecast patient conditions into the future. ML techniques can include one or more of the following features: executing automated time-series process segmentation includes processing each of the two or more time-series data sequences using a deep learning (DL) model provided as one of a bidirectional long short-term memory (LSTM) sequence classifier using supervised learning, and an autoencoder using unsupervised learning; the at least one time-series transformation includes one or more of a temporal transformation, a spectral transformation, a shape transformation, a statistical transformation, an autoencoder transformation, and a decomposition transformation, a pass-through transformation, processing each feature data set through a reinforcement learning framework, among others.

Turning briefly to FIG. 3, for purposes of illustration, some examples of categories/classifications of input variables or variable types 300 are listed. These variable types 300 can include: (a) persona variables 310—for example, age, weight, BMI before pregnancy, marital status, education, working status of mother, monthly income level, birthplace of mother, ethnicity of mother, ethnicity of partner, partner's age, partner's education, partner's working status, marital satisfaction, smoking status of mother, smoking status of partner, alcohol consumption frequency of mother, physical activity of mother, and mother disability (status and type); (b) pregnancy variables 320 unplanned/planned pregnancy, trimester, vitamin B6 level, gestational diabetes status, HbA1c level, average hours of sleep in current week, anxiety level (scaled 1 to 5), delivery fear (scaled 1 to 5), pregnancy nausea, previous childbirth history, depressive symptoms in third trimester, fetus growth rate, and average blood pressure in current week during pregnancy; (c) childbirth variables 330-delivery week, mode of delivery (C-section or natural), risky pregnancy status (emergency procedures, etc.), miscarriage, stillbirth, child birth length, and birth complications; (d) infant variables 340-birthweight, baby staying in incubator after birth, baby gender, premature status, baby abnormality status, hepatitis+within one month of birth, baby sleep cycle, and breastfeeding; (e) postpartum variables 350-BMI after delivery, postpartum (PP) vitamin B6 level, PP TSH level, PP gestational diabetes status, PP HbA1c level, average hours of sleep post-delivery, and average blood pressure in current week (post-delivery); and (f) psychometric variables 360-depression history, anti-depressants history, PMS, negative attitude toward baby, gender reluctance of mother for baby, gender reluctance of partner for baby, social support, partner help with baby, and domestic violence.

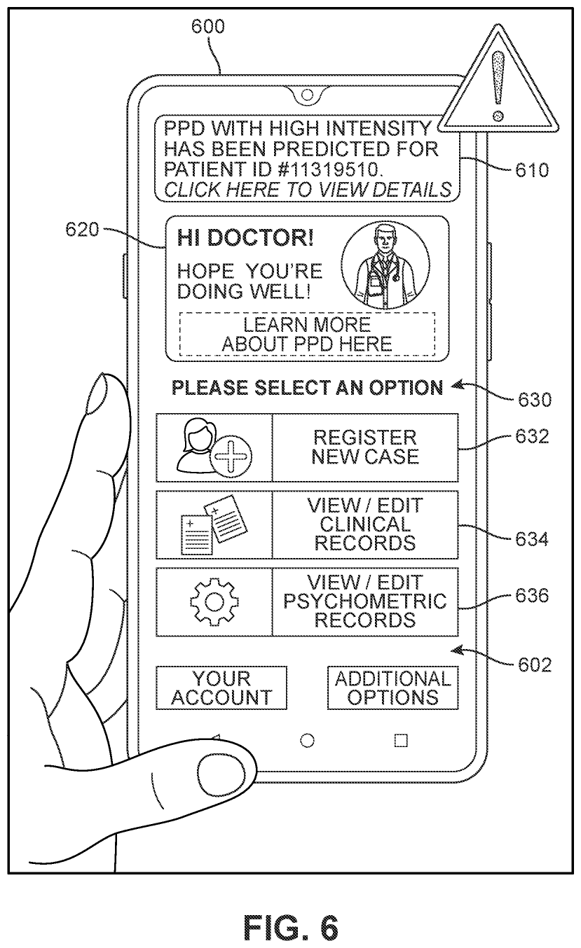

Returning to FIG. 2, it can be appreciated that based on the role of the user (e.g., patient 262, doctor 264, medical facility 266, etc.) can play an important factor in determining what kind of information should be shared from the system 250 to the user, and what kind of information should be requested or accepted by the system 250 by the user. Thus, in different embodiments, back-end module 242 of the app 240 can include role rules that define what type of information and presentation of information, as well as authentication, should be required and applied when exchanging patient-related data and analyses through user interface 260. For example, in the case of medical professionals, the standard of authentication can be significantly higher than for the patient, as the professionals may be allowed to access records for multiple patients for whom privacy is vital. Furthermore, as will be discussed in greater detail with reference to FIGS. 6, 7, and 8, the system can be configured to vary the options and user interface modes made available to the users based on the role rules. Other role rules can be defined for mental health professionals, health care workers, home care workers, babysitters, family members, etc. that will determine how the app 240 will respond to and present information from the user.

In different embodiments, the system 250 can be configured to maintain an active, and interactive, dialogue and/or exchange of information with the end-user in order to collect data needed to generate an accurate prediction. As noted earlier, the prediction model 270 can rely on time-bound rules, which make the collection of data at specific intervals helpful to the performance of the PPD/mental health risk prediction process. Thus, in some embodiments, the system 250 includes intake manager 230, which automatically generates-via an intake generator 232-a request for the specific data items that are identified as missing and are desired for the given week (or other time frame). In one example, each week the intake generator 232 can auto-generate a questionnaire that is presented to the patient via user interface 260. This questionnaire can be a self-reported series of questions that can be answered via the user interface 260 and shown to the user at their mobile device. In some embodiments, the questionnaire can assess cognitive, behavioral, affective, and somatic symptoms of depression, as well as sleeping/eating disturbances, anxiety/insecurity, emotional liability, cognitive impairment, loss of self, guilt/shame, and contemplating harming oneself, questions that are targeted to a specific population, ethnicity, or community, questions that are targeted for specific time periods in the pre-natal and post-natal experience. In some embodiments, standard questionnaire forms or portions thereof (e.g., the Beck Depression Inventory (BDI), the Edinburgh Postnatal Depression Scale (EPDS), the Postpartum Depression Screening Scale (PDSS), Structured Clinical Interview for DSM-IV (SCID), etc.) may be incorporated.

In addition, a data collection prompter 234 of the intake manager 230 can be configured to identify, with reference to a scheduling database, recurring or otherwise key dates or periods at which clinical/lab data (or other data that may not be effectively collected via the user interface) should be provided. For example, the vitamin B6 level should be monitored regularly, and the system 250 can generate reminders and requests to the patient, doctor, and/or medical facility to encourage testing and reporting compliance. The data collection prompter 234 can be configured to transmit a signal to the app 240 that identifies the target user(s), and the information being requested. As just a few non-limiting examples, the data collection prompter 234 can require that the user interface 260 present one or more reminders to the patient 262 that there is an upcoming lab event to schedule or an alert that a lab event was missed, a notification to the doctor 264 that a particular patient of theirs has missed a scheduled lab event and suggest a follow-up, and a notification to the medical facility 266 that a patient has not yet scheduled an appointment for bloodwork that needs to be performed at week 25, and a request to contact the patient to schedule the appointment. In other words, in different embodiments, each target user (user type) can receive notifications with different content and requirements that nevertheless involve the same activity or event for the same patient.

In some embodiments, the user interface can employ or be supplemented by a digital assistant or other automated chatbot (“chatbot”) or automated agent. As a general matter, such an assistant should be understood to encompass a processing environment that is adapted to utilize spoken or typed natural language to interact with and respond to an end-user. In some embodiments, some or all of the processing environment may be referred to as, included in, and/or include the digital assistant. Furthermore, a digital assistant and associated systems for communicating with a digital assistant may include one or more user devices, such as a computer, a server, a database, and a network. For example, a digital assistant running on a server could communicate with a user over a network.

In different embodiments, the digital assistant may be accessed via the user interface 260. Throughout this application, an “interface” may be understood to refer to a mechanism for communicating content through a client application to an application user. In some examples, interfaces may include pop-up windows that may be presented to a user via native application user interfaces (UIs), controls, actuatable interfaces, interactive buttons or other objects that may be shown to a user through native application UIs, as well as mechanisms that are native to a particular application for presenting associated content with those native controls. In addition, the terms “actuation” or “actuation event” refers to an event (or specific sequence of events) associated with a particular input or use of an application via an interface, which can trigger a change in the display of the application. Furthermore, a “native control” refers to a mechanism for communicating content through a client application to an application user. For example, native controls may include actuatable or selectable options or “buttons” that may be presented to a user via native application UIs, touch-screen access points, menus items, or other objects that may be shown to a user through native application UIs, segments of a larger interface, as well as mechanisms that are native to a particular application for presenting associated content with those native controls. The term “asset” refers to content that may be presented in association with a native control in a native application. As some non-limiting examples, an asset may include text in an actuatable pop-up window, audio associated with the interactive click of a button or other native application object, video associated with a therapeutic interface, or other such information presentation.

Thus, throughout the pregnancy and postpartum periods, data can continuously and/or regularly be received at the system 250 and ingested by the prediction model 270. In different embodiments, the prediction model 270 may determine there is sufficient data to make a prediction with a high degree of confidence. It should be understood that the prediction can be modified from week to week as additional data is obtained by the system 250. In cases where no PPD or other mental health diagnosis is predicted (at least for the time being) for the given patient, no action need be taken. However, if the prediction model 270 generates a PPD or mental health risk prediction for a patient, the response selection module 290 of system 250 is notified. In some embodiments, a participant alert system 294 can immediately be triggered and cause a presentation of an alert message to be shown in the doctor or other medical professional's app 240 (or app 240) notifying them of the prediction. In one embodiment, the response selection module 290 can also automatically select one or more actions that should be performed, based on whether the patient is still pregnant or has already given birth, from a recommended action database 292. The recommended action database 292 identifies a plurality of strategies that are also time-based. These actions can be presented via participant alert system 294 to the doctor 264 and/or medical facility 266 to increase intelligent and timely responses to the potential PPD experience for the patient. In some embodiments, the system 250 can gently describe one or more recommended actions to the patient 262 (e.g., try speaking to family, friends, counselors, doctors), without necessarily indicating to the patient that they have PPD. In other words, it is important that the final mental health diagnosis remain in the hands of the medical professionals treating the patient. In some embodiments, the patient may have identified one or more persons in an available support network 226 during their registration with the system 250, and—depending on the permissions given to the system 250 by the patient—the system 250 can be configured to automatically present a message to one or more of the persons in the available support network 226 asking them to check in on the patient.

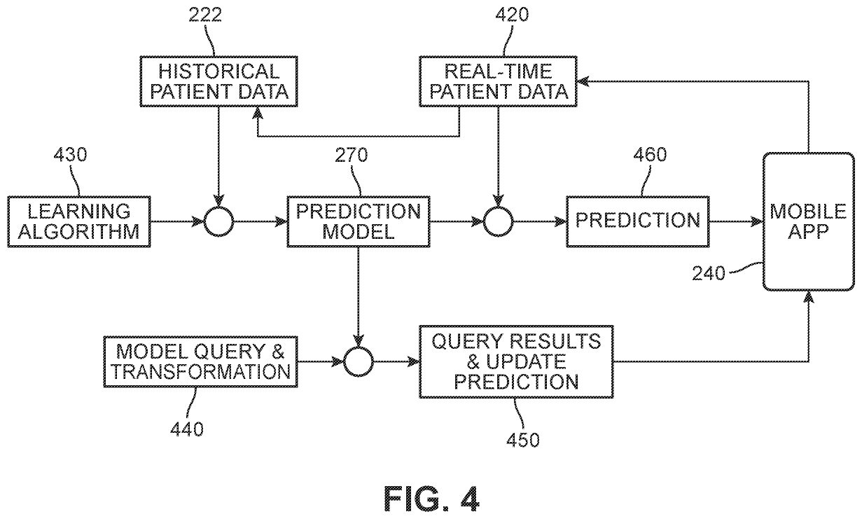

Moving now to FIG. 4, a schematic diagram illustrates an example of the development and implementation of the prediction model 270. As shown in FIG. 4, as real-time or near-real-time patient data 420 (e.g., from diagnosed PPD patients as well as undiagnosed women who are pregnant or given birth, and/or experiencing other mental health conditions) is received for individual women, it can be stored as part of the historical patient data 222, as discussed with reference to FIG. 2. The prediction model 270 further incorporates a learning algorithm 430 that is continuously reworked and adjusted as the new data is received, and improving its performance at each iteration. In addition, as noted earlier, in different embodiments, the prediction model 270 operates as a probabilistic time-bound model, where the predictions will dynamically adapt to the time slot, window, duration, or period that the individual patient is entering or is reached. These windows of time can be predefined durations that may be regularly spaced (e.g., daily, weekly, monthly, etc., such that a first time slot refers to the first week of the pregnancy, a second time slot refers to the second week of the pregnancy, a third time slot refers to the third week of the pregnancy, then similarly week by week after the pregnancy until one year post-partum has been reached). In other examples, these windows of time can be predefined durations that are irregularly spaced (e.g., such that a first time slot refers to the first two months of the pregnancy, a second time slot refers to the three weeks immediately following, a third time slot refers to the four days immediately following those three weeks, etc.). The windows of time can be defined based on the historical patient data 222 and ongoing research in the area of PPD that can indicate a likely pattern of experiences and symptoms and how they occur in the timeline.

In different embodiments, as a first set of data (e.g., corresponding to a first window of time) is received for a particular patient via the app 240, an initial prediction 460 may be generated by the prediction model 270. In some embodiments, these predictions can be produced automatically in response to incoming data. In other embodiments, a user may submit a query 440 to the prediction model 270 requesting an updated prediction that relies on new data that may have been obtained during the time since the previous prediction. For example, over time, the prediction model 270 can be transformed and predictions will be updated 450 to represent the most up-to-date data as additional sets of data are received. These predictions can then be shared with the appropriate end-user class associated with the patient (e.g., to the doctor for the patient who has been predicted to have PPD, but not, in most cases, to the patient directly) via the app 240.

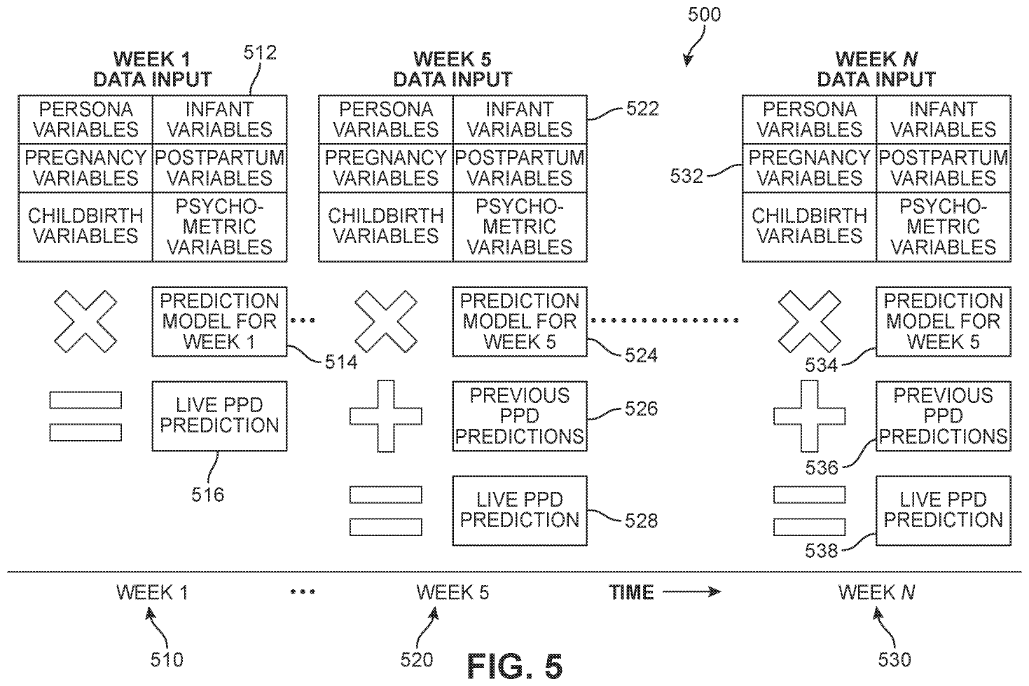

Moving to FIG. 5, an example that schematically represents a time-bound nature of the prediction model is presented via a model timeline diagram 500 (or diagram 500). In the diagram 500, it is shown that, for each week of pregnancy and for the weeks of the year following childbirth, data can be collected or otherwise obtained about the patient. In different embodiments, a plurality of standard inputs including data associated with some or all of the variable types discussed in FIG. 3 are received. It can be appreciated that the weekly data can include missing values when appropriate. Thus, a first set 512 of data for or during “Week 1” stage 510 of a woman's pregnancy, includes values related to the persona variables, pregnancy variables, and psychometric variables, while infant variables, postpartum variables, and childbirth variables may be empty or blank (e.g., “non-applicable” or N/A). The first set 512 is inputted into a first prediction model 514 that applies Week 1 weights to the variables in order to generate a first mental health prediction (“first prediction”) 516 based only on the Week 1 stage 510.

For each week subsequent to Week 1, a similar process is performed, except the historical data of previous weeks is also incorporated into the prediction. For example, a second set 522 of data for or during “Week 5” stage 520 of a woman's pregnancy, includes values related to the persona variables, pregnancy variables, and psychometric variables, while infant variables, postpartum variables, and childbirth variables may continue to be empty or blank (e.g., “non-applicable” or N/A). Although for purposes of clarity a “Week 5” is depicted, it should be understood that in different embodiments, the patient's data would be collected regularly in the intervening weeks (Week 2, Week 3, Week 4) and separate live PPD predictions also generated for each week. It can further be appreciated that some data items may be constant, such as some of the persona variables (e.g., demographic information), and only modified in a given week if the patient indicates there has been a change or correction.

In different embodiments, the second set 522 can then be inputted into a second prediction model 524 that can differ from the operation of the first prediction model 514 in that a different arrangement/assignment of weights can be attached to the variables. In some embodiments, the difference in weights can be minor, or only some of the variables may be weighted differently, while other variables' weights remain the same. This change in weights based on the patient's time phase (what week is it) is done to optimize the performance of the model based on current PPD research and indicators of the relative significance of some variables in the PPD diagnosis at some time periods, and the increasing or decreasing significance of those same variables at a later (different) time period. Weights can also be modified in response to ongoing model training and testing that lead to improvements in the model's accuracy. Thus, the second prediction model 524 can receive the second set 522, and along with the previous PPD predictions (before Week 5) 526 and relevant historical data for the patient, generate a second mental health prediction (“second prediction”) 528 for the Week 5 stage 520. In some embodiments, weights may be fine-tuned as new training data or anonymized patient data, as well as feedback for past predictions, are received by the system. These adjustments can reflect the expanding source of knowledge and lead to improved model performance and prediction accuracy over time. As an example, a new training dataset can be received that indicates the weights assigned to specific demographic-related variables should be greater, while the weights assigned to specific hormonal levels (or some other variable) should be less.

As the pregnancy proceeds, the patient can continue to submit data. For purposes of simplicity, a third set 532 of data for or during a generic “Week n” stage 530 of a woman's pregnancy or postpartum year includes values related to the persona variables, and psychometric variables. If this data is based on information obtained while the woman is still pregnant, pregnancy variables can also be included, while infant variables, postpartum variables, and childbirth variables may continue to be empty or blank (e.g., “non-applicable” or N/A). On the other hand, if the data is based on information obtained after delivery, infant variables, postpartum variables, and childbirth variables can be collected in addition to the persona variables and psychometric variables, while pregnancy variables can be empty or blank (e.g., “non-applicable” or N/A).

In different embodiments, the third set 532 can then be inputted into a third prediction model 534 that differs from both the first prediction model 514 and the second prediction model 524 in that a different arrangement of weights can be attached to the variables, as described above. Thus, the third prediction model 534 can receive the third set 532, and along with the previous PPD predictions (before Week n) 536 and relevant historical data for the patient, generate a third mental health prediction (“third prediction”) 538 for the Week n stage 530. This time-bound operation of the prediction model is employed to significantly improve the confidence level for each prediction and ensure that a patient's potential for PPD or another mental health disorder is monitored continuously, as well as bringing the end-users' attention to the importance of ongoing, regular checks across an extended timeframe, and thereby greatly improving the likelihood that patients receive more timely care and treatment.

Simply for purposes of illustration, one example of a set of weights associated with a set of variables is included in Table 1 below. It can be appreciated that for different weeks of the pregnancy and the postpartum period, the weight assigned to a given variable can be modified to reflect the time-bound nature of its significance in the prediction at that time. In this example, the weights may be assigned a value in the range from zero to one, where values closer toward zero indicate the variable has less of an impact on the prediction for that week, and values closer toward one indicate the variable has more of an impact on the prediction for that week. It is understood that the number (e.g., more or less variables) of variables and combination of variable types may be modified in different embodiments.

TABLE 1

| | | | Pre- | | | |
|---|---|---|---|---|---|---|
| Weight | | Smoking | mature | | Vit | |
| Factor | | Status of | Birth | Baby | B6 | HbA1C |
| In | Age | Mother | Status | Gender | level | level |
| Week 5 of | If age is | 1 | 0 | 0 | 0.6 | 0.6 |
| pregnancy | between 24- | | | | | |
| | 36 → 0.5 | | | | | |
| | If age is <24 | | | | | |
| | and >36 → 1 | | | | | |

. . .

| | | | | | | |
|---|---|---|---|---|---|---|
| Week 18 | If age is | 1 | 0 | If | 1 | 1 |
| of | between 24- | | | known | | |
| pregnancy | 36 → 0.5 | | | → 0.6 | | |
| | If age is <24 | | | If un- | | |
| | and >36 → 1 | | | known | | |
| | | | | → 0 | | |

. . .

| | | | | | | |
|---|---|---|---|---|---|---|
| Week 3 | If age is | 1 | 1 | 0.8 | 0.6 | 1 |
| post- | between 24- | | | | | |
| partum | 36 → 0.5 | | | | | |
| | If age is <24 | | | | | |
| | and >36 → 1 | | | | | |

. . .

FIGS. 6, 7, and 8 depict examples of user interfaces that may be provided based on the end-user class type (e.g., doctor, patient, medical facility, etc.). In FIG. 6, an example of a first user interface experience (“first interface”) 602 for the app is provided to a first user via a first computing device 600 associated with a doctor-type user. In this case, the display presents a welcome header 620 (“Hi Doctor!/Hope you're doing good!/Learn more about PPD here”) that allows the doctor to review the characteristics and current research targeting PPD, as well as a set of doctor-specific selectable options 630 such as a first doctor option 632 (“Register new case”), a second doctor option 634 (“View/Edit Clinical Records”), and a third doctor option 636 (“View/Edit Psychometric Records”). These options are shown as examples only, and it should be appreciated that different or additional options can also be offered (e.g., app settings, communications with other healthcare providers regarding patients, treatment plans developed for patients, recommended actions, past predictions for patients, etc.) as well as user account information. Selection of each option can open a new window or page in the app or a webpage link that enables access to the selected feature. In some embodiments, whenever the system determines a patient has a high likelihood of being diagnosed with PPD, an immediate, real-time notification or alert 610 can be pushed to the doctor's device, or shown in the app. The alert 610 in this example indicates a current patient needs to be reviewed (“PPD with high intensity has been predicted for Patient ID #11319510. Click here to view details”) and whose information can be accessed quickly (shortcut) by clicking on the alert message.

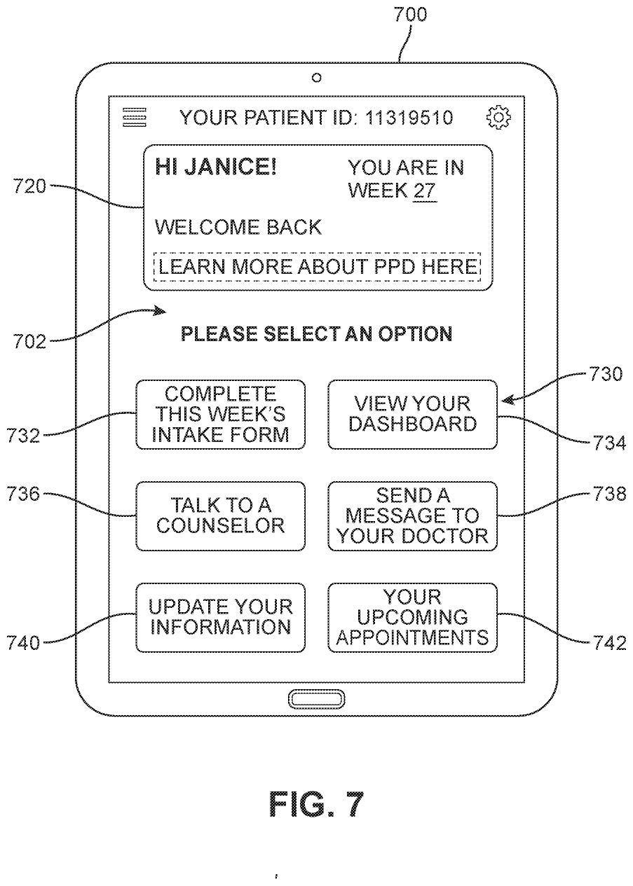

In FIG. 7, an example of a second user interface experience (“second interface”) 702 for the app is provided to a second user via a second computing device 700 associated with a patient-type user. In this case, the display presents a welcome header 720 (“Hi [Patient Name]!/Welcome back!/You are in Week 27”) that allows the patient to view information about PPD and their timeline, as well as a set of patient-specific selectable options 730 such as a first patient option 732 (“Complete this week's intake form” to provide the current data set, including questionnaires and other non-laboratory-data), a second patient option 734 (“View Your Dashboard” that allows the patient to see their historical data and assessments), a third patient option 736 (“Talk to a counselor” enabling the patient to request an appointment with a registered mental health provider, and/or be provided access to a mental health counselor via the app in real-time), a fourth patient option 738 (“Send a message to your doctor” to communicate with their doctor via the app, with messages that can be accessed by the doctor via their own version of the app), a fifth patient option 740 (“Update your information”), and a sixth patient option 742 (“Your upcoming appointments” with reminders about important time-based data collection events and sessions). These options are shown as examples only, and it should be appreciated that different or additional options can also be offered (e.g., app settings, communications with other healthcare providers regarding their care, their current treatment plan, and scheduling or rescheduling appointments, etc.) as well as user account information. Selection of each option can open a new window or page in the app or a webpage link that enables access to the selected feature.

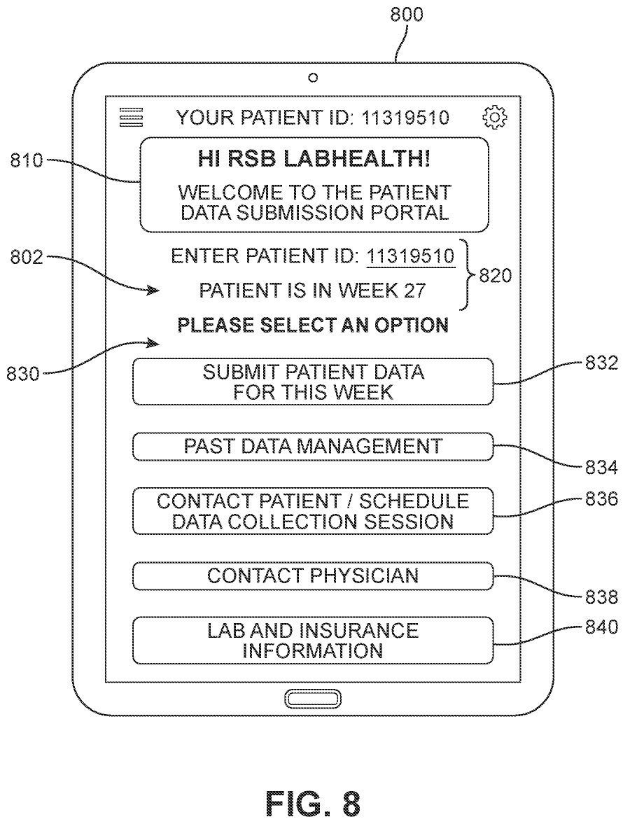

In FIG. 8, an example of a third user interface experience (“third interface”) 802 for the app is provided to a third user via a third computing device 800 associated with a medical facility-type user. In this case, the display presents a welcome header 810 (“Hi [Facility Name]!/Welcome to the Patient Data Submission Portal”) that allows the personnel of the facility to view information about the registered monitored patients for whom they are responsible to collect data or perform various laboratory work. In this example, a patient selection mode 820 is shown, in which the patient's ID is entered, allowing the personnel's access to a set of facility-specific selectable options 830 for the selected patient, including as a first facility option 832 (“Submit patient data for this week” to provide the most recent/current data set for the selected patient including labwork), a second facility option 834 (“Past Data management” that allows the facility to review the patient's historical data and update, flag, or correct the data), a third facility option 836 (“Contact patient/Schedule data collection session” enabling the facility to request an appointment to perform labwork or other data collection, and/or contact the patient via the app in real-time), a fourth facility option 838 (“Contact the physician” to communicate with the selected patient's doctor via the app, with messages that can be accessed by the doctor via their own version of the app), and a fifth facility option 840 (“Lab and insurance information” to view or update insurance and other records associated with the selected patient). These options are shown as examples only, and it should be appreciated that different or additional options can also be offered (e.g., app settings, communications with other healthcare providers regarding the patients' care, the patient's expected lab needs, and scheduling or rescheduling appointments, etc.) as well as user account information. Selection of each option can open a new window or page in the app or a webpage link that enables access to the selected feature.

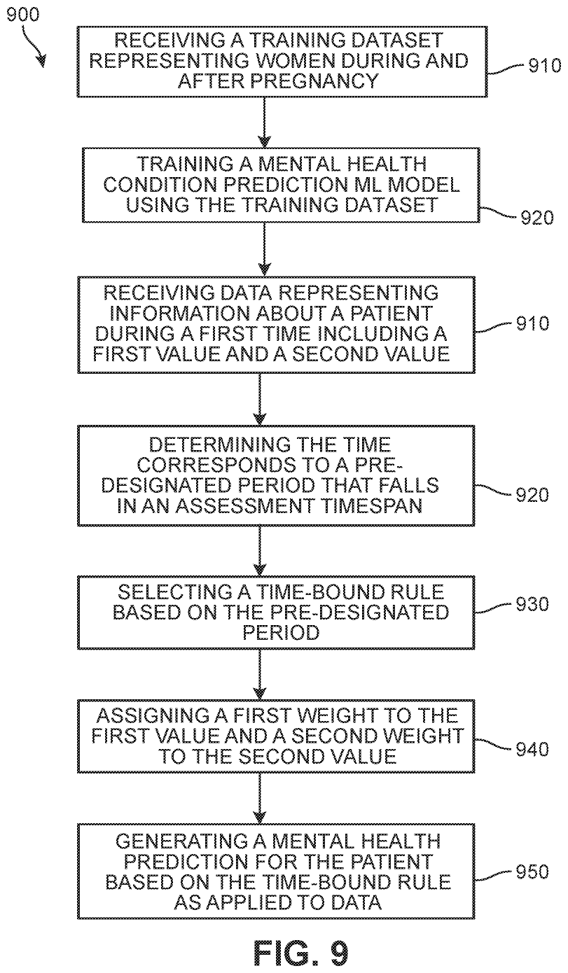

FIG. 9 is a flow chart illustrating an embodiment of a method 900 of predicting a mental health condition during and/or after a life event. The method 900 includes a first step 910 of receiving, at a mental health risk assessment system, a first training dataset including data representing women during and in the year following pregnancy, and a second step 920 of training, using the first training dataset, a mental health condition prediction machine learning (ML) model to generate mental health risk predictions based on one or more time-bound rules. The method 900 also includes a third step 930 of receiving, at a mental health risk assessment system, a first set of data representing information about a first patient during a first time, the first set of data including a first value for a first variable and a second value for a second variable, and a fourth step 940 of determining the first time corresponds to a first pre-designated period that falls in an assessment timespan when a person is either pregnant or within a year after experiencing childbirth. In addition, a fifth step 950 includes selecting a first time-bound rule based on the first pre-designated period, and a sixth step 960 includes assigning, at the mental health condition prediction ML model and based on the first time-bound rule, a first weight to the first value and a second weight to the second value. Furthermore, a seventh step 970 includes generating, via the mental health condition prediction ML model, a first prediction for the first patient based on the first time-bound rule as applied to the first set of data, and an eighth step 980 includes automatically transmitting a notification describing the first prediction to one or more health personnel associated with the first patient.

In other embodiments, the method may include additional steps or aspects. In another example, the method further includes a step of segmenting the assessment timespan into a series of weeks, where the first pre-designated period is one of the weeks in the series of weeks. In another embodiment, the method can also include steps of receiving, at the mental health risk assessment system, a second set of data representing information about the first patient during a second time, the second set of data including a third value for the first variable and a fourth value for the second variable, determining the second time corresponds to a second pre-designated period that falls in the assessment timespan, selecting a second time-bound rule based on the second pre-designated period, assigning, at the mental health condition prediction ML model and based on the second time-bound rule, a third weight to the third value and a fourth weight to the second value, the third weight differing from the first weight, and generating, via the mental health condition prediction ML model, a second prediction for the first patient based at least on the second set of data that differs from the first prediction. In one embodiment, the second prediction is further based on historical data that includes the first set of data.

In different embodiments, the method can also include steps of receiving, at the mental health risk assessment system, a first registration for the first patient at a second time, determining the first patient is in a second pre-designated period that falls in the assessment timespan, selecting a first intake form based on the second pre-designated period, and automatically presenting, via a user interface for an application of the mental health risk assessment system shown on a display for a computing device, the first intake form. In some embodiments, the method can include steps of receiving, at the mental health risk assessment system, a first registration for the first patient at a second time, determining the first patient is in a second pre-designated period that falls in the assessment timespan, selecting a first laboratory test based on the second pre-designated period, and automatically presenting, via a user interface for an application of the mental health risk assessment system shown on a display for a computing device, a notification reminding the first patient to schedule the first laboratory test.

In another example, the method includes automatically presenting, at a display of a computing device for a medical facility associated with the first patient (e.g., via a user interface for the application of the system, or a push notification, text message, pop-up window, SMS, etc.), a notification reminding the medical facility to submit results of the first laboratory test for the first patient. In some embodiments, the method includes automatically presenting, at a display of a computing device (e.g., via a user interface for the application of the system, or a push notification, text message, pop-up window, SMS, etc.) for a medical professional associated with the first patient, the first prediction. In some embodiments, the first prediction indicates a high likelihood of PPD occurring in the first patient, and the method further comprises precluding presentation of the first prediction to the first patient via the application.

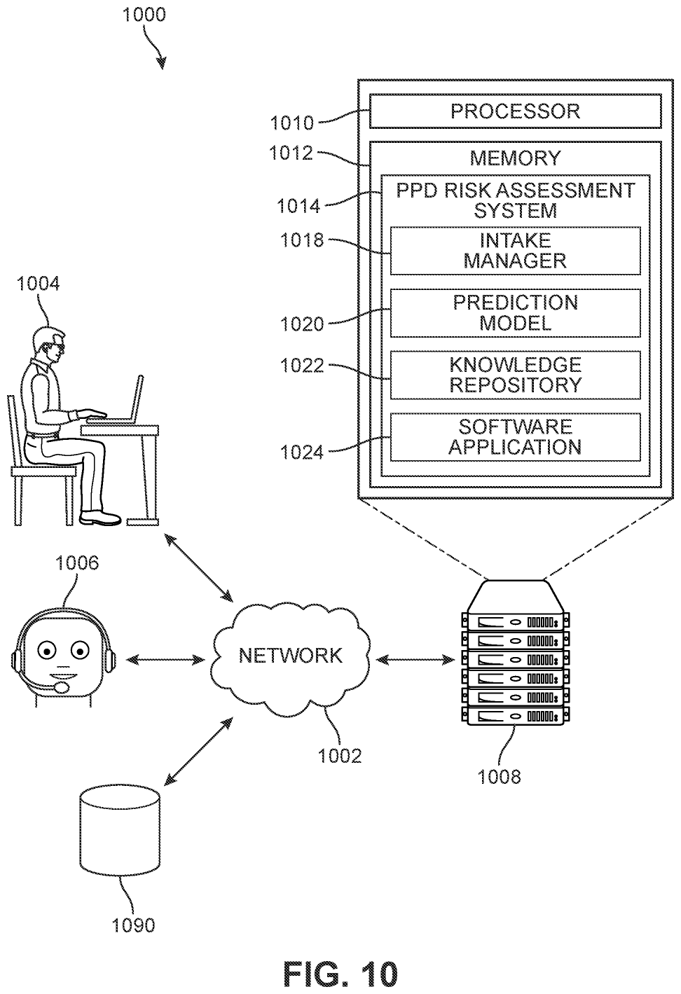

FIG. 10 is a schematic diagram of an environment 1000 for a mental health risk assessment system 1014 (or system 1014), according to an embodiment. The environment 1000 may include a plurality of components capable of performing the disclosed methods. For example, environment 1000 includes a user device 1004, a computing/server system 1008, and a database 1090. The components of environment 1000 can communicate with each other through a network 1002. For example, user device 1004 may retrieve information from database 1090 via network 1002. In some embodiments, network 1002 may be a wide area network (“WAN”), e.g., the Internet. In other embodiments, network 1002 may be a local area network (“LAN”).

As shown in FIG. 10, components of the system 1014 may be hosted in computing system 1008, which may have a memory 1012 and a processor 1010. Processor 1010 may include a single device processor located on a single device, or it may include multiple device processors located on one or more physical devices. Memory 1012 may include any type of storage, which may be physically located on one physical device, or on multiple physical devices. In some cases, computing system 1008 may comprise one or more servers that are used to host the system.

While FIG. 10 shows one user device, it is understood that one or more user devices may be used. For example, in some embodiments, the system may include two or three user devices. In some embodiments, the user device may be a computing device used by a user. For example, user device 1004 may include a smartphone or a tablet computer. In other examples, user device 1004 may include a laptop computer, a desktop computer, and/or another type of computing device. The user devices may be used for inputting, processing, and displaying information. Referring to FIG. 10, environment 1000 may further include database 1090, which stores test data, training data, parameters and weights, classification data, variable-risk relationship data, recommendation (response) database (corpus), aspects of the knowledge repository, and/or other related data for the components of the system as well as other external components. This data may be retrieved by other components for system 1014. As discussed above, system 1014 may include an intake manager 1018, a prediction model 1020, a knowledge repository 1022, and a software application 1024. Software application 1024 may work in conjunction with a digital assistant 1006 to promote intelligent communication and enable automated questions and answers with the end-users. Each of these components may be used to perform the operations described herein.

For purposes of this application, an “interface” may be understood to refer to a mechanism for communicating content through a client application to an application user. In some examples, interfaces may include pop-up windows that may be presented to a user via native application user interfaces (UIs), controls, actuatable interfaces, interactive buttons/options or other objects that may be shown to a user through native application UIs, as well as mechanisms that are native to a particular application for presenting associated content with those native controls. In addition, the terms “actuation” or “actuation event” refers to an event (or specific sequence of events) associated with a particular input or use of an application via an interface, which can trigger a change in the display of the application. Furthermore, a “native control” refers to a mechanism for communicating content through a client application to an application user. For example, native controls may include actuatable or selectable options or “buttons” that may be presented to a user via native application UIs, touch-screen access points, menus items, or other objects that may be shown to a user through native application UIs, segments of a larger interface, as well as mechanisms that are native to a particular application for presenting associated content with those native controls. The term “asset” refers to content that may be presented in association with a native control in a native application. As some non-limiting examples, an asset may include text in an actuatable pop-up window, audio associated with the interactive click of a button or other native application object, video associated with the user interface, or other such information presentation.

It should be understood that the text, images, and specific application features shown in the figures are for purposes of illustration only and in no way limit the manner by which the application may communicate or receive information. In addition, in other embodiments, one or more options or other fields and text may appear differently and/or may be displayed or generated anywhere else on the screen(s) associated with the client's system, including spaced apart from, adjacent to, or around the user interface. In other words, the figures present only one possible layout of the interface, and do not in any way limit the presentation arrangement of any of the disclosed features.

Embodiments may include a non-transitory computer-readable medium (CRM) storing software comprising instructions executable by one or more computers which, upon such execution, cause the one or more computers to perform the disclosed methods. Non-transitory CRM may refer to a CRM that stores data for short periods or in the presence of power such as a memory device or Random Access Memory (RAM). For example, a non-transitory computer-readable medium may include storage components, such as, a hard disk (e.g., a magnetic disk, an optical disk, a magneto-optic disk, and/or a solid state disk), a compact disc (CD), a digital versatile disc (DVD), a floppy disk, a cartridge, and/or a magnetic tape.

To provide further context, in some embodiments, some of the processes described herein can be understood to operate in a system architecture that can include a plurality of virtual local area network (VLAN) workstations at different locations that communicate with a main data center with dedicated virtual servers such as a web server for user interfaces, an app server for OCR and data processing, a database for data storage, etc. As a general matter, a virtual server is a type of virtual machine (VM) that is executed on a hardware component (e.g., server). In some examples, multiple VMs can be deployed on one or more servers.

In different embodiments, the system may be hosted at least in part in a cloud computing environment offering ready scalability and security. The cloud computing environment can include, for example, an environment that hosts the document processing management service. The cloud computing environment may provide computation, software, data access, storage, etc. services that do not require end-user knowledge of a physical location and configuration of system(s) and/or device(s) that hosts the policy management service. For example, a cloud computing environment may include a group of computing resources (referred to collectively as “computing resources” and individually as “computing resource”). It is contemplated that implementations of the present disclosure can be realized with appropriate cloud providers (e.g., AWS provided by Amazon™, GCP provided by Google™, Azure provided by Microsoft™, etc.).

The methods, devices, and processing described above may be implemented in many different ways and in many different combinations of hardware and software. For example, all or parts of the implementations may be circuitry that includes an instruction processor, such as a Central Processing Unit (CPU), microcontroller, or a microprocessor; or as an Application Specific Integrated Circuit (ASIC), Programmable Logic Device (PLD), or Field Programmable Gate Array (FPGA); or as circuitry that includes discrete logic or other circuit components, including analog circuit components, digital circuit components or both; or any combination thereof.

While various embodiments of the invention have been described, the description is intended to be exemplary, rather than limiting, and it will be apparent to those of ordinary skill in the art that many more embodiments and implementations are possible that are within the scope of the invention. Accordingly, the invention is not to be restricted except in light of the attached claims and their equivalents. Also, various modifications and changes may be made within the scope of the attached claims.

We claim:

1. A method for predicting a mental health condition during and/or after pregnancy, the method comprising:
receiving, at a mental health risk assessment system, a first training dataset including data representing women during and in the year following pregnancy;
training, using the first training dataset, a mental health condition prediction machine learning (ML) model to generate mental health risk predictions based on one or more time-bound rules;
receiving, at a mental health risk assessment system, a first set of data representing information about a first patient during a first time, the first set of data including a first value for a first variable and a second value for a second variable;
determining the first time corresponds to a first pre-designated period that falls in an assessment timespan when a person is either pregnant or within a year after experiencing childbirth;
selecting a first time-bound rule based on the first pre-designated period;
assigning, at the mental health condition prediction ML model and based on the first time-bound rule, a first weight to the first value and a second weight to the second value;
generating, via the mental health condition prediction ML model, a first prediction for the first patient based on the first time-bound rule as applied to the first set of data; and
automatically transmitting a notification describing the first prediction to one or more health personnel associated with the first patient.

2. The method of claim 1, further comprising segmenting the assessment timespan into a series of weeks, wherein the first pre-designated period is one of the weeks in the series of weeks.

3. The method of claim 1, further comprising:
receiving, at the mental health risk assessment system, a second set of data representing information about the first patient during a second time, the second set of data including a third value for the first variable and a fourth value for the second variable;
determining the second time corresponds to a second pre-designated period that falls in the assessment timespan;
selecting a second time-bound rule based on the second pre-designated period;
assigning, at the mental health condition prediction ML model and based on the second time-bound rule, a third weight to the third value and a fourth weight to the second value, the third weight differing from the first weight; and
generating, via the mental health condition prediction ML model, a second prediction for the first patient based at least on the second set of data that differs from the first prediction.

4. The method of claim 3, wherein the second prediction is further based on historical data that includes the first set of data.

5. The method of claim 1, further comprising:
receiving, at the mental health risk assessment system, a first registration for the first patient at a second time;
determining the first patient is in a second pre-designated period that falls in the assessment timespan;
selecting a first intake form based on the second pre-designated period; and
automatically presenting, via a user interface for an application of the mental health risk assessment system shown on a display for a computing device, the first intake form.

6. The method of claim 1, further comprising:
receiving, at the mental health risk assessment system, a first registration for the first patient at a second time;
determining the first patient is in a second pre-designated period that falls in the assessment timespan;
selecting a first laboratory test based on the second pre-designated period; and
automatically presenting, via a user interface for an application of the mental health risk assessment system shown on a display for a computing device, a notification reminding the first patient to schedule the first laboratory test.

7. The method of claim 6, further comprising automatically presenting, at a display of a computing device for a medical facility associated with the first patient, a notification reminding the medical facility to submit results of the first laboratory test for the first patient.

8. The method of claim 1, further comprising automatically presenting, at a display of a computing device for a medical professional associated with the first patient, the first prediction.

9. The method of claim 8, wherein the first prediction indicates a high likelihood of PPD occurring in the first patient, and the method further comprises precluding presentation of the first prediction to the first patient via an application.

10. A non-transitory computer-readable medium storing software comprising instructions executable by one or more computers which, upon such execution, cause the one or more computers to predict a mental health condition during and/or after pregnancy by performing the following:
receive, at a mental health risk assessment system, a first training dataset including data representing women during and in the year following pregnancy;
train, using the first training dataset, a mental health condition prediction machine learning (ML) model to generate mental health risk predictions based on one or more time-bound rules;
receive, at a mental health risk assessment system, a first set of data representing information about a first patient during a first time, the first set of data including a first value for a first variable and a second value for a second variable;
determine the first time corresponds to a first pre-designated period that falls in an assessment timespan when a person is either pregnant or within a year after experiencing childbirth;
select a first time-bound rule based on the first pre-designated period;
assign, at the mental health condition prediction ML model and based on the first time-bound rule, a first weight to the first value and a second weight to the second value;
generate, via the mental health condition prediction ML model, a first prediction for the first patient based on the first time-bound rule as applied to the first set of data; and
automatically transmit a notification describing the first prediction to one or more health personnel associated with the first patient.

11. The non-transitory computer-readable medium storing software of claim 10, wherein the instructions further cause the one or more computers to:
receive, at the mental health risk assessment system, a second set of data representing information about the first patient during a second time, the second set of data including a third value for the first variable and a fourth value for the second variable;
determine the second time corresponds to a second pre-designated period that falls in the assessment timespan;
select a second time-bound rule based on the second pre-designated period;
assign, at the mental health condition prediction ML model and based on the second time-bound rule, a third weight to the third value and a fourth weight to the second value, the third weight differing from the first weight; and
generate, via the mental health condition prediction ML model, a second prediction for the first patient based at least on the second set of data that differs from the first prediction.

12. The non-transitory computer-readable medium storing software of claim 11, wherein the second prediction is further based on historical data that includes the first set of data.

13. The non-transitory computer-readable medium storing software of claim 10, wherein the instructions further cause the one or more computers to:
receive, at the mental health risk assessment system, a first registration for the first patient at a second time;
determine the first patient is in a second pre-designated period that falls in the assessment timespan;
select a first intake form based on the second pre-designated period; and
automatically present, via a user interface for an application of the mental health risk assessment system shown on a display for a computing device, the first intake form.

14. The non-transitory computer-readable medium storing software of claim 10, wherein the instructions further cause the one or more computers to:
receive at the mental health risk assessment system, a first registration for the first patient at a second time;
determine the first patient is in a second pre-designated period that falls in the assessment timespan;
select a first laboratory test based on the second pre-designated period; and
automatically present, via a user interface for an application of the mental health risk assessment system shown on a display for a computing device, a notification reminding the first patient to schedule the first laboratory test.

15. The non-transitory computer-readable medium storing software of claim 14, wherein the instructions further cause the one or more computers to present, at a display of a computing device for a medical facility associated with the first patient, a notification reminding the medical facility to submit results of the first laboratory test for the first patient.

16. A system for predicting a mental health condition during and/or after pregnancy, the system comprising one or more computers and one or more storage devices storing instructions that are operable, when executed by the one or more computers, to cause the one or more computers to:
receive, at a mental health risk assessment system, a first training dataset including data representing women during and in the year following pregnancy;
train, using the first training dataset, a mental health condition prediction machine learning (ML) model to generate mental health risk predictions based on one or more time-bound rules;
receive, at a mental health risk assessment system, a first set of data representing information about a first patient during a first time, the first set of data including a first value for a first variable and a second value for a second variable;
determine the first time corresponds to a first pre-designated period that falls in an assessment timespan when a person is either pregnant or within a year after experiencing childbirth;
select a first time-bound rule based on the first pre-designated period;
assign, at the mental health condition prediction ML model and based on the first time-bound rule, a first weight to the first value and a second weight to the second value;
generate, via the mental health condition prediction ML model, a first prediction for the first patient based on the first time-bound rule as applied to the first set of data; and
automatically transmit a notification describing the first prediction to one or more health personnel associated with the first patient.

17. The system of claim 16, wherein the instructions further cause the one or more computers to:
receive, at the mental health risk assessment system, a first registration for the first patient at a second time;
determine the first patient is in a second pre-designated period that falls in the assessment timespan;
select a first laboratory test based on the second pre-designated period; and
automatically present, via a user interface for an application of the mental health risk assessment system shown on a display for a computing device, a notification reminding the first patient to schedule the first laboratory test.

18. The system of claim 17, wherein the instructions further cause the one or more computers to automatically present, at a computing device for a medical facility associated with the first patient, a notification reminding the medical facility to submit results of the first laboratory test for the first patient.

19. The system of claim 16, wherein the instructions further cause the one or more computers to automatically present, at a display of a computing device for a medical professional associated with the first patient, the first prediction.

20. The system of claim 19, wherein the first prediction indicates a high likelihood of PPD occurring in the first patient, and the instructions further cause the one or more computers to preclude presentation of the first prediction to the first patient via an application.
